(19) United States
(12) Patent Application Publication
(10) Pub. No.: US 2015/0217088 A1
(43) Pub. Date: Aug. 6, 2015

(54) METHOD AND DEVICE FOR INTERVENTIONAL SITE MANAGEMENT OF SHEATHS AND CATHETERS

(71) Applicant: Lakeshore Medical Innovations, LLC, Byron Center, MI (US)

(72) Inventors: Mark E. Zyzelewski, Kalamazoo, MI (US); Brian O. Tierney, Grand Rapids, MI (US); Sean R. Collins, Rockford, MI (US); Jihad A. Mustapha, Ada, MI (US)

(21) Appl. No.: 14/682,347

(22) Filed: Apr. 9, 2015

Related U.S. Application Data
(63) Continuation-in-part of application No. PCT/US13/63802, filed on Oct. 8, 2013.
(60) Provisional application No. 61/711,533, filed on Oct. 9, 2012.
(60) Provisional application No. 62/100,125, filed on Jan. 6, 2015.

(51) Int. Cl. A61M 25/02; A61M 25/06; A61B 17/00; A61B 17/34; A61B 19/10

(52) U.S. Cl. CPC A61M 25/02; A61B 17/3415; A61B 19/10; A61B 17/3423; A61B 17/00234; A61M 25/0662; A61B 2019/103; A61B 2017/3492; A61B 2017/3419; A61B 2017/00243; A61B 2017/00951; A61M 2025/0681; A61M 2025/026

(57) ABSTRACT

A method of managing an access cite and site management device for an interventional cardiology access lumen having a cylindrical hub and a tubular portion extending from the cylindrical hub includes adhering an adhesive pad directly or indirectly to a patient at an intervention cardiology access site. After the adhering, an access lumen is deployed and a portion of the access lumen positioned against a retention pad that is supported by the adhesive pad. The retention pad has a surface configured to the portion of the access lumen.

### CROSS REFERENCE TO RELATED APPLICATIONS

This application is a continuation-in-part of International Patent Cooperation Treaty Application No. PCT/US2013/063802, filed on Oct. 8, 2013, which claims the benefit of U.S. provisional patent application Ser. No. 61/711,533, filed on Oct. 9, 2012; and this application claims the benefit of U.S. provisional patent application Ser. No. 62/100,125, filed on Jan. 6, 2015, the disclosures of which are hereby incorporated herein by reference in their entireties.

### FIELD OF USE

This invention is in the field of devices used to secure and properly retain sheaths and catheters that may be used during an interventional catheterization procedure.

### BACKGROUND OF THE INVENTION

During every interventional catheterization case there are sheaths and catheters that are used to gain access to the patient's vasculature. Once access is achieved, the sheaths and catheters are used to introduce fluids and devices directly into the patient vasculature. The devices may include, but are not limited to, guide wires, catheters, angioplasty balloons, and stent delivery systems. The sheaths and catheters remain in the patient's artery throughout the entire procedure, and multiple devices can be inserted and removed during the procedure. While inserting and removing devices, it is not uncommon for the sheath to move with the movement of the device. This movement is not ideal and can cause the sheath to slide out of position. The sheath can move farther into the artery or slide out of the artery depending on the movement. Either situation can create issues which negatively affect the procedure.

If the sheath moves farther into the artery, the vessel can spasm and contract, making it difficult to insert and remove devices. The spasms can also make it difficult to remove the sheath. If the sheath loses its position by moving out of the vessel, artery access can be completely lost.

Currently, to prevent the movement of the sheaths and catheters, tape is used to retain the sheath. Once artery access is gained, the sheath is taped to the skin to attempt to retain its position. Typically, the tape used is a polyurethane skin dressing. This process of using the dressing can be cumbersome. The dressing completely covers the sheath opening and must be modified to allow access to the proximal end, or opening, of the sheath. The modification of the dressing can weaken the structure of the dressing, and reduce the effectiveness during the repeated movements of devices through the sheath.

Another issue is the adhesion of the dressing to the access site and sheath throughout the procedure. During the procedure, there typically is blood that may seep from the access site and/or proximal end of the sheath. The adhesives used on the dressings do not perform as well when exposed to fluids. The continued exposure to the fluids throughout the procedure can weaken the bond of the dressing to the sheath and the skin, and therefore lead to unwanted movement of the sheath.

### SUMMARY OF THE INVENTION

The present invention is a securement device and method whose goal is to securely retain the access lumen, such as a sheath or catheter, throughout the procedure without being affected by movement of the devices through the sheath. The device also has features which help maintain the adhesion of the device to the skin when fluids are seen during the procedure. The device is also designed to fit with various access lumen types.

A method of managing an access site and site management device for an interventional cardiology access lumen having a cylindrical hub and a tubular portion extending from the cylindrical hub, according to an aspect of the invention, includes adhering an adhesive pad directly or indirectly to a patient at an intervention cardiology access site. After the adhering, an access lumen is deployed and a portion of the access lumen positioned against a retention pad that is supported by the adhesive pad. The retention pad has a surface configured to the portion of the access lumen. The access lumen is retained to the retention pad with a top retention strip.

The retention pad surface configuration may have a receiving geometry complimentary to that of the hub. Typically, the retention pad surface will have a partial-circular shape configured to the size and shape of the cylindrical hub of the access lumen. The retention pad surface configuration may include side ribs that are configured to the size and shape of a side port of the access lumen. The side ribs may extend laterally in opposite directions from the partial-circular shape to allow the side port of the access lumen to extend in either lateral direction.

Proximal access may be provided to an insertion hole in the access lumen that is positioned against the retention pad and retained by the top retention strip. The top retention strip may be an adhesive strip. The top retention strip may define a through-channel and the method includes passing an extension of the side port through the channel. The adhesive pad may include another through-channel that aligns with the through-channel in the top retention strip and the extension of the side port may pass through both through-channels.

The adhesive pad may be adhered directly to the patient by wrapping the adhesive pad at least partially around the patient's extremity, such as the wrist or ankle. The adhesive pad may be adhered indirectly to the patient by adhering the adhesive pad to a drape at the intervention site. Access may be provided to an insertion opening at the hub of the retained access lumen.

One embodiment of the invention is to make an access site management device which fits with all components from all sheath manufacturers. There are a few features that are common on all interventional sheaths. One feature is a round hub for insertion of interventional devices. The hub is cylindrical in shape with the proximal end of the hub having a hole for insertion of devices, and the distal end of the hub attaching to the tubular sheath which is inserted into the patient's artery. The other feature that is common is a side port attached to the hub of the sheath. The side port is used for injection fluids into the patient. The side port exits the hub normal to the axis of the sheath. These common features allow for a consistent, rigid attachment to a securement device. The securement device incorporates an overall circular cross-sectioned retention pad. This circular sectioned pad allows for engagement with the cylindrical hub of the sheath. Protruding normal to the axis of the circular section is a rib which engages with the side port of the sheath. Ideally, this rib contains a front and back side, which captures the side port, and prevents the movement of the sheath along the axis of the sheath. Therefore, as devices are moved in and out of the sheath, the sheath is held rigidly and prevents any movement.

Another embodiment of the access site management device is the unique adhesive attachment. To prevent the inadvertent movement of the sheath during use, the securement device incorporates a circumferential adhesive strip which adheres to the skin at the access site and circumferentially to both sides of the device. This circumferential adhesion ensures strong adhesive bonds to the skin even if the access site and sheath encounter seeping blood. The device also incorporates a top adhesive which covers the sheath hub and side port, and ensures containment in the circular cross-sectioned pad. With the circular cross-sectioned pad with side ribs, and the adhesive bonds, any interventional sheath can be held rigidly throughout a procedure.

An interventional cardiology access lumen site management device for use with an access lumen having a cylindrical hub and a tubular portion extending from said cylindrical hub, according to an aspect of the invention, includes an adhesive pad that is adapted to adhere directly or indirectly to a patient. A retention pad is supported with the adhesive pad. The retention pad has a surface configuration that is adapted to engage a portion of an access lumen and adapted to retain the access lumen. The site preparation device is adapted to adhere directly or indirectly prior to deployment of the access lumen and is adapted to secure the access lumen after deployment of the access lumen. A side port receiver is configured to the size and shape of a side port of the access lumen.

These and other objects, advantages and features of this invention will become apparent upon review of the following specification in conjunction with the drawings.

### BRIEF DESCRIPTION OF THE DRAWINGS

FIG. 1 is a perspective view of a sheath that is useful with the invention;

FIG. 2 is a top plan view of the access site management device;

FIG. 3 is a perspective view of the access site management device that is partially deployed showing engagement of the sheath side port in the device side channel;

FIG. 4 is a perspective view of the access site management device that is fully deployed showing the adhesive attachment to the skin;

FIG. 5 is the same view as FIG. 4 taken from a different direction of the access site management device showing the adhesive attachment to the sheath with exposure of the proximal end of the sheath;

FIG. 6 is a top plan view of an alternative embodiment of an access site management device;

FIG. 7 is the same view as FIG. 3 of the embodiment in FIG. 6;

FIG. 8 is a perspective view of another alternative embodiment taken from the top, front and right side thereof;

FIG. 9 is a perspective view of the embodiment in FIG. 8 taken from the top, front and left side thereof;

FIG. 10 is a perspective view of the embodiment in FIG. 8 taken from the bottom front and right side thereof;

FIG. 11 is a perspective view of the embodiment in FIG. 8 used with a method of managing an access site;

FIG. 12 is the same view as FIG. 11 illustrating replacement of an interventional sheath;

FIG. 13 is the same view as FIG. 12 showing unlocking of the side port;

FIG. 14 is the same view as FIG. 12 showing locking of the new side port;

FIG. 15 is the same view as FIG. 8 of another alternative embodiment;

FIG. 16 is a side elevation view taken from the left side of the embodiment in FIG. 15;

FIG. 17 is a front elevation view of the embodiment in FIG. 15;

FIG. 18 is a perspective view of the embodiment in FIG. 15 used with a method of managing an access site;

FIG. 19 is a perspective view of the method in FIG. 18 taken from a different direction than that of FIG. 18;

FIG. 20 is the same view as FIG. 11 of yet another alternative embodiment;

FIG. 21 is a view of the embodiment in FIG. 20 taken from a different direction than the view in FIG. 20; and

FIG. 22 is the same view as FIG. 20 illustrating assembly of the device to the sheath.

### DESCRIPTION OF THE PREFERRED EMBODIMENT

The present invention is embodied in an access site management device and method whose goal is to securely retain the sheath throughout the procedure without being affected by movement of devices through the sheath.

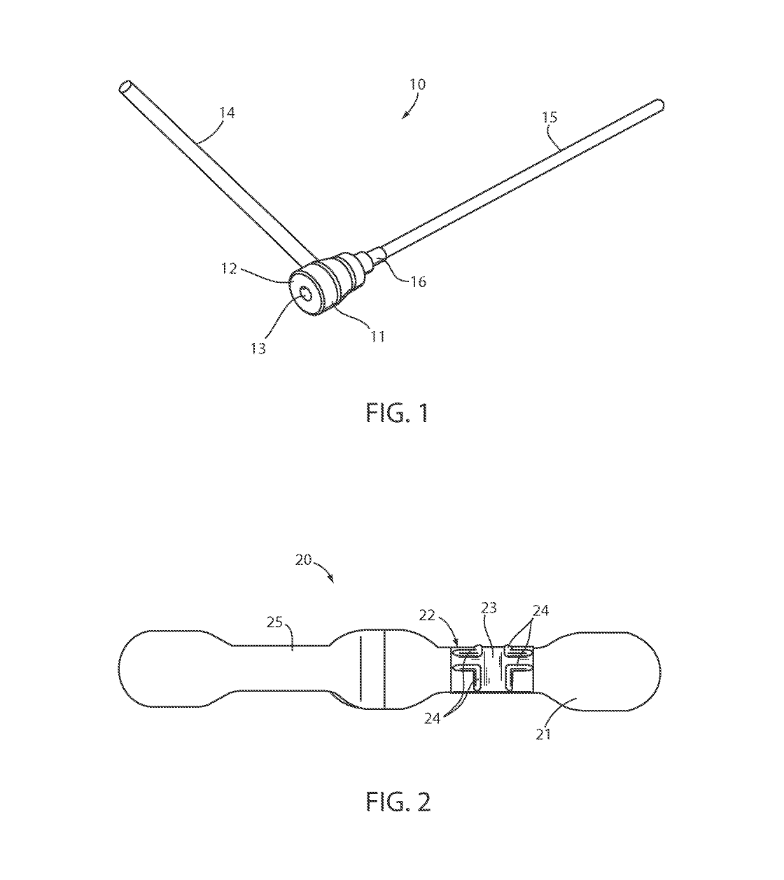

FIG. 1 shows a typical interventional sheath 10. A typical interventional sheath 10 contains a cylindrical hub 11, a proximal side 12 with an insertion hole 13. The sheath 10 has a side port 14 which exits the hub 11 normal to the axis of the hub. The sheath 10 has a tubular portion 15 which extends from the distal side 16 of the cylindrical hub 11.

FIG. 2 shows a top view of the securement device 20. The securement device 20 has a circumferential adhesive pad 21 which can be directly connected with the patient by a wrap around an extremity, such as the wrist or ankle of a patient. Alternatively, adhesive pad 21 can be indirectly connected with the patient, such as by attachment of the pad to a drape around the procedure site. Attached to the adhesive pad 21 is the partial-circular cross-sectioned retention pad 22. The retention pad 22 contains a partial-circular section 23 for locating and holding the hub of the sheath. The retention pad 22 also contains side ribs 24 which locate and hold the side port of the sheath. The securement device 20 also contains a top retention strip 25 for containing the sheath when placed into the retention pad. Retention strip 25 may be retained by adhesive, Velcro, clips, or the like. In the illustrated embodiment, retention strip 25 is retained by adhesive.

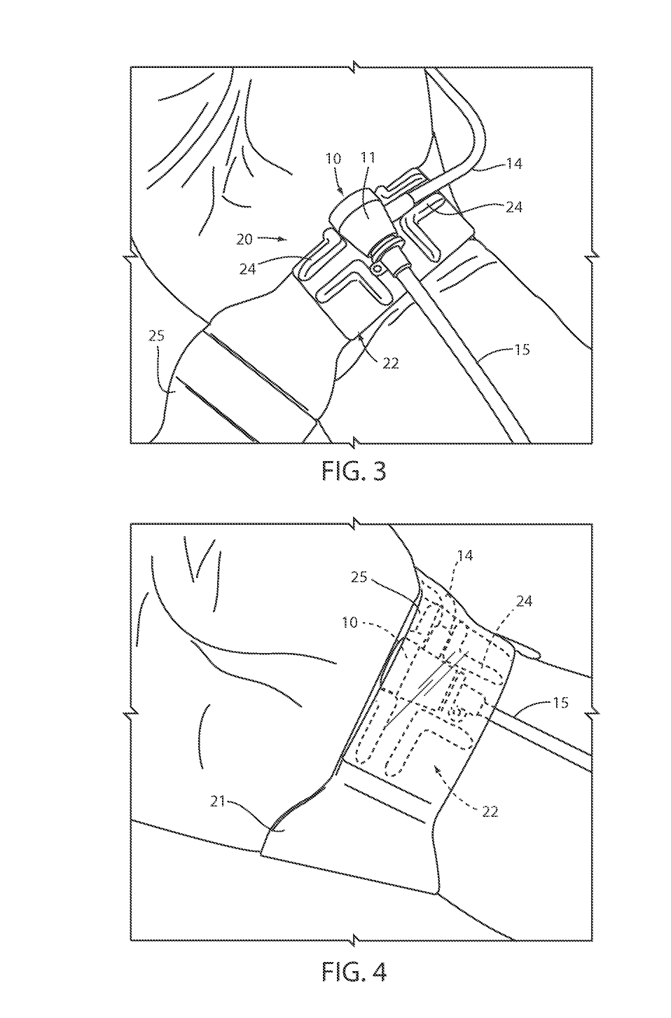

FIG. 3 shows a perspective view of the securement device 20 with adhesive pad 21 retained to the patient's wrist. The adhesive pad can be attached to the patient prior to deploying the access lumen 10 thereby avoiding adherence problems from trying to apply adhesive over a site that has blood or other fluids present. Once adhesive pad 21 is attached to the patient directly or indirectly, sheath 10 is then inserted in the patient's vessel and cylindrical hub 11 is installed in the retention pad 22. The figure shows the engagement of the sheath hub 11 in retention pad 22, and the engagement of the side port 14 with the side ribs 24 of the retention pad 22.

FIG. 4 shows the application of retention strip 25 to retain sheath 10 to retention pad 22 after circumferential adhesion of the adhesive pad 21 around the wrist of the patient and deployment of sheath 10.

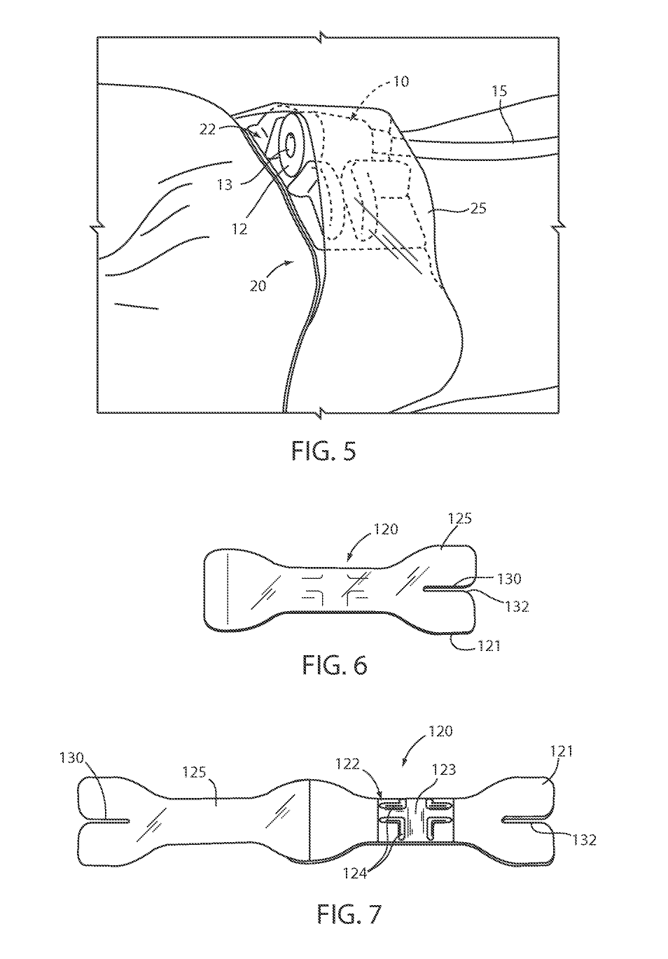

FIG. 5 shows the securement device 20 with the sheath 10 installed in the retention pad 22. The figure shows the top adhesive 25 covering and containing the sheath 10 in the retention pad 22. The figure shows the open access to the insertion hole 13 in the proximal side 12 of the sheath for access to insertion hole 13 during the interventional cardiology procedure.

An alternative embodiment 120 shown in FIGS. 6 and 7, of an interventional cardiology access lumen site management device has the same features as site management device 20 except that it has an adhesive pad 121 that is similar to adhesive pad 21 except that it has a through-channel 132 defined therein. Site management device 120 further has a top retention strip 125 that is similar to top retention device 25 to retain a stent against a retention pad (not shown) that is similar to retention pad 22 with a partial circular surface (not shown) that is similar to section 23 formed therein. However, top retention strip 125 has a through-channel 130 that aligns with through-channel 132. Through-channels 130, 132 accommodate a tubular extension of side port 14 of stent 10 to allow more freedom of movement of the extension of side port 14.

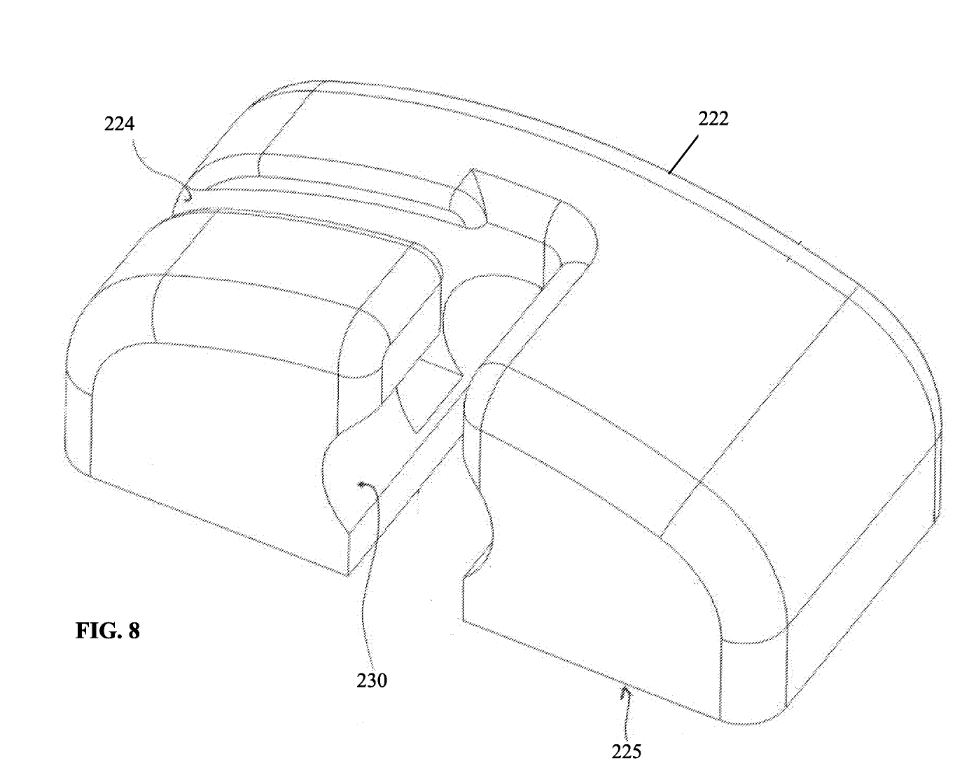

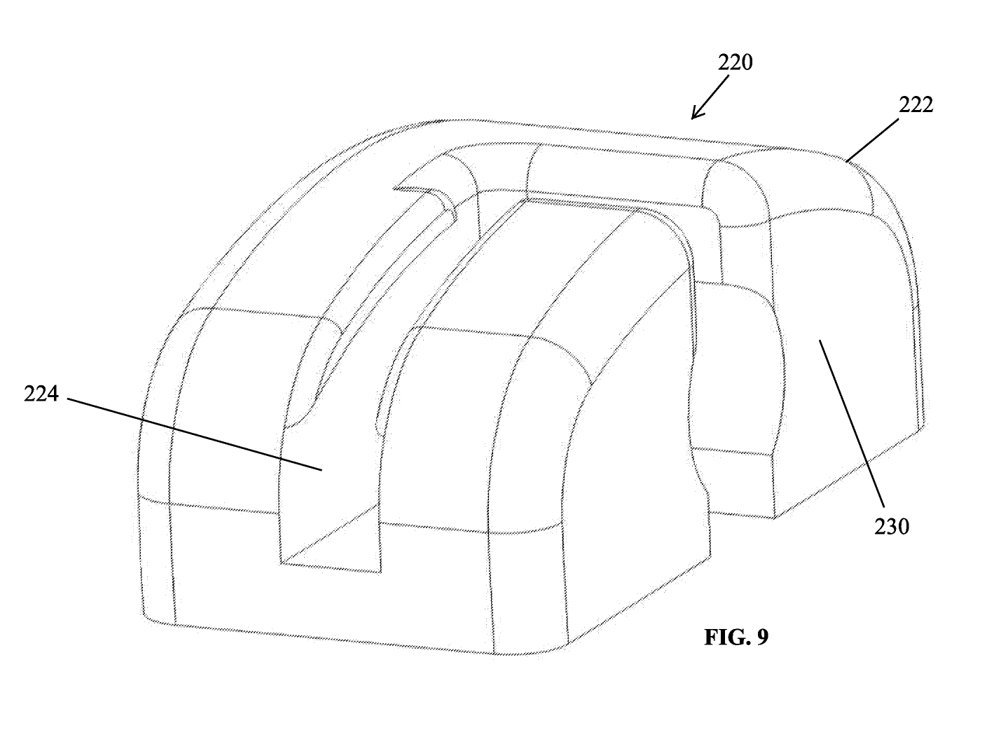

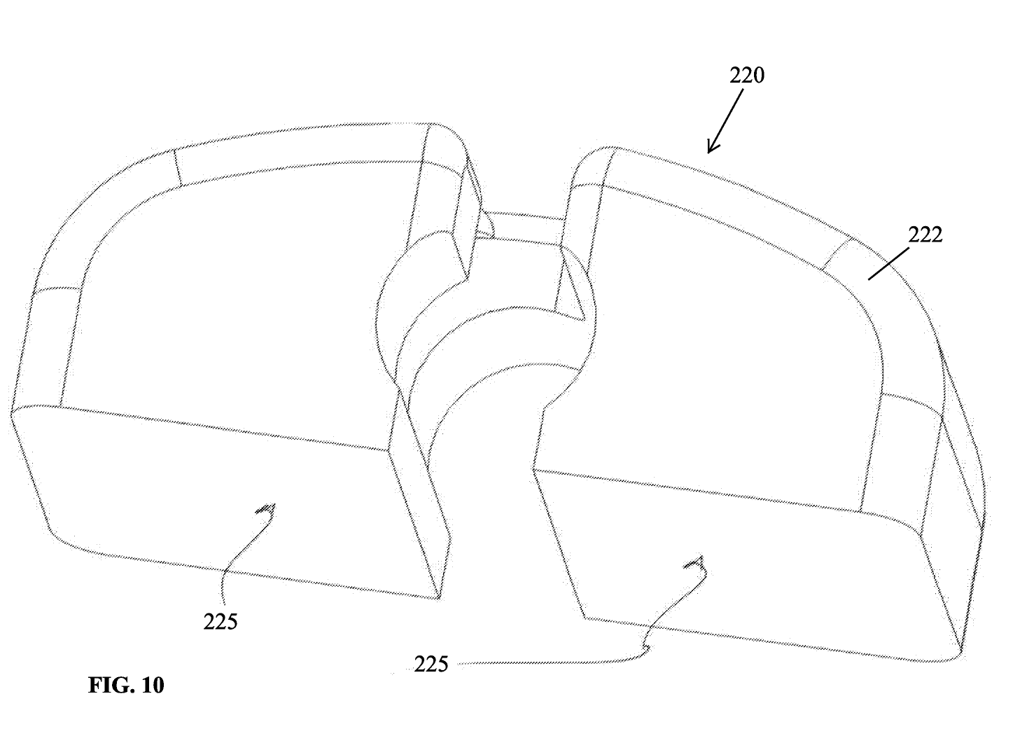

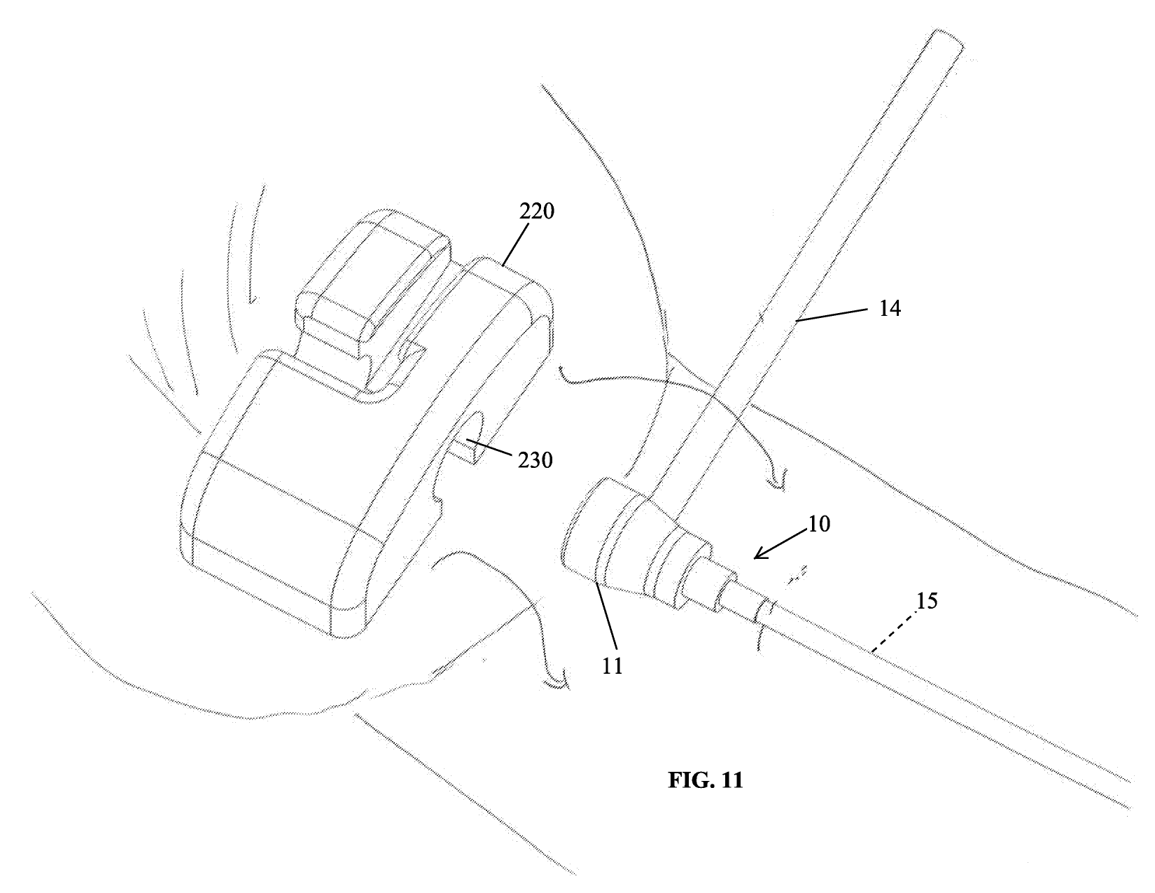

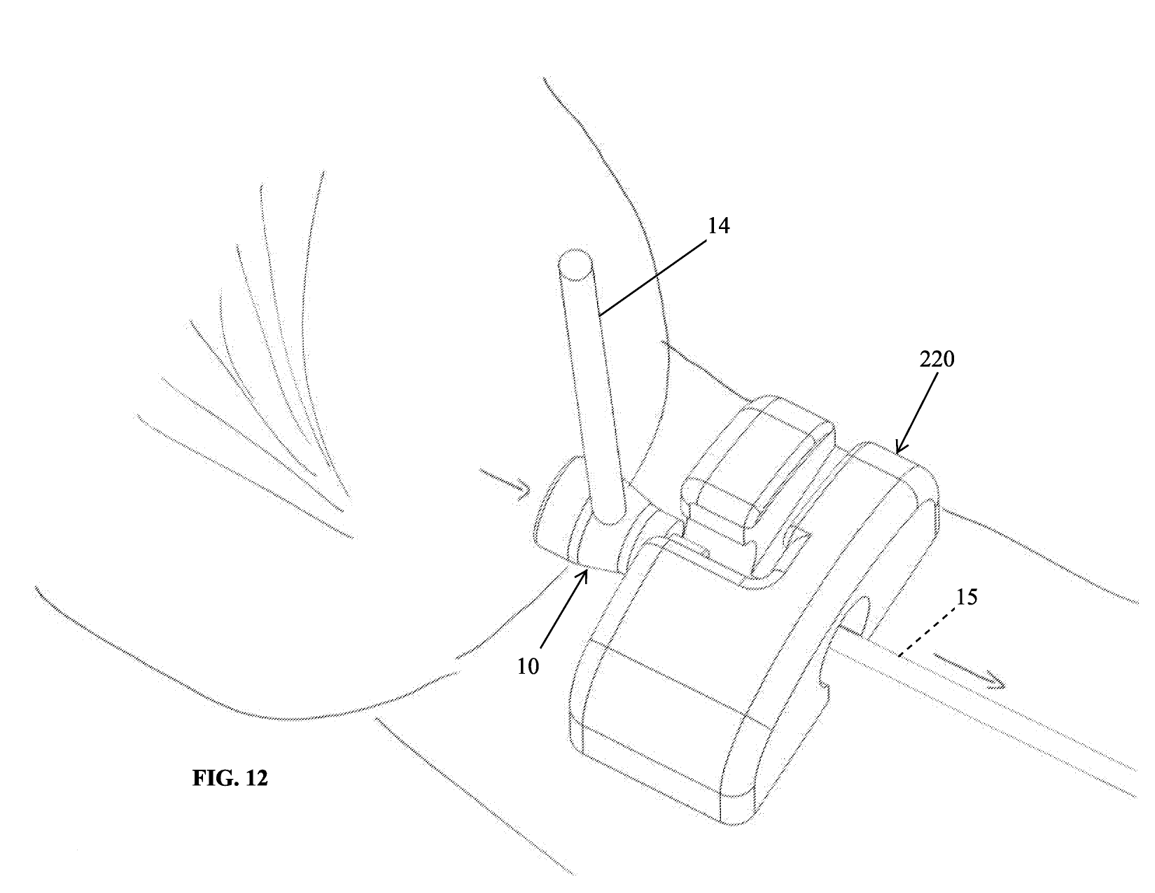

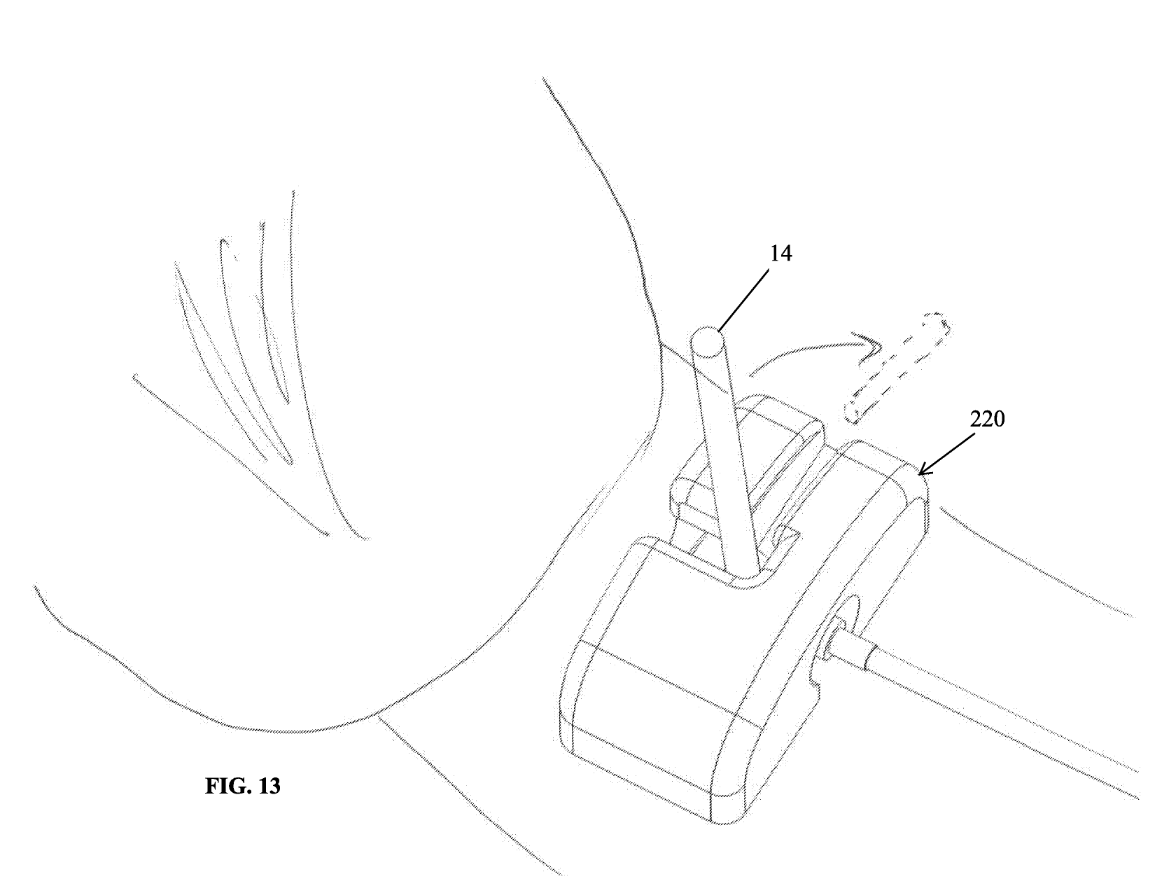

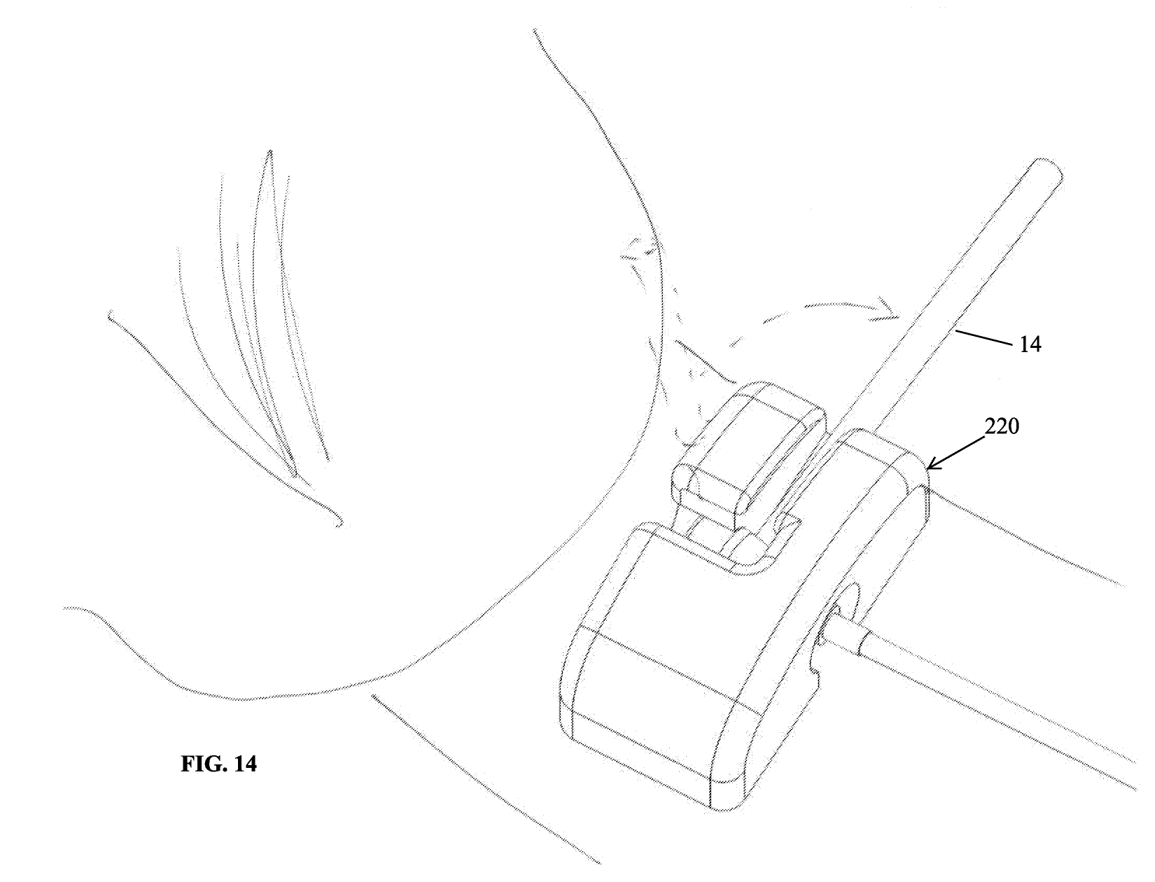

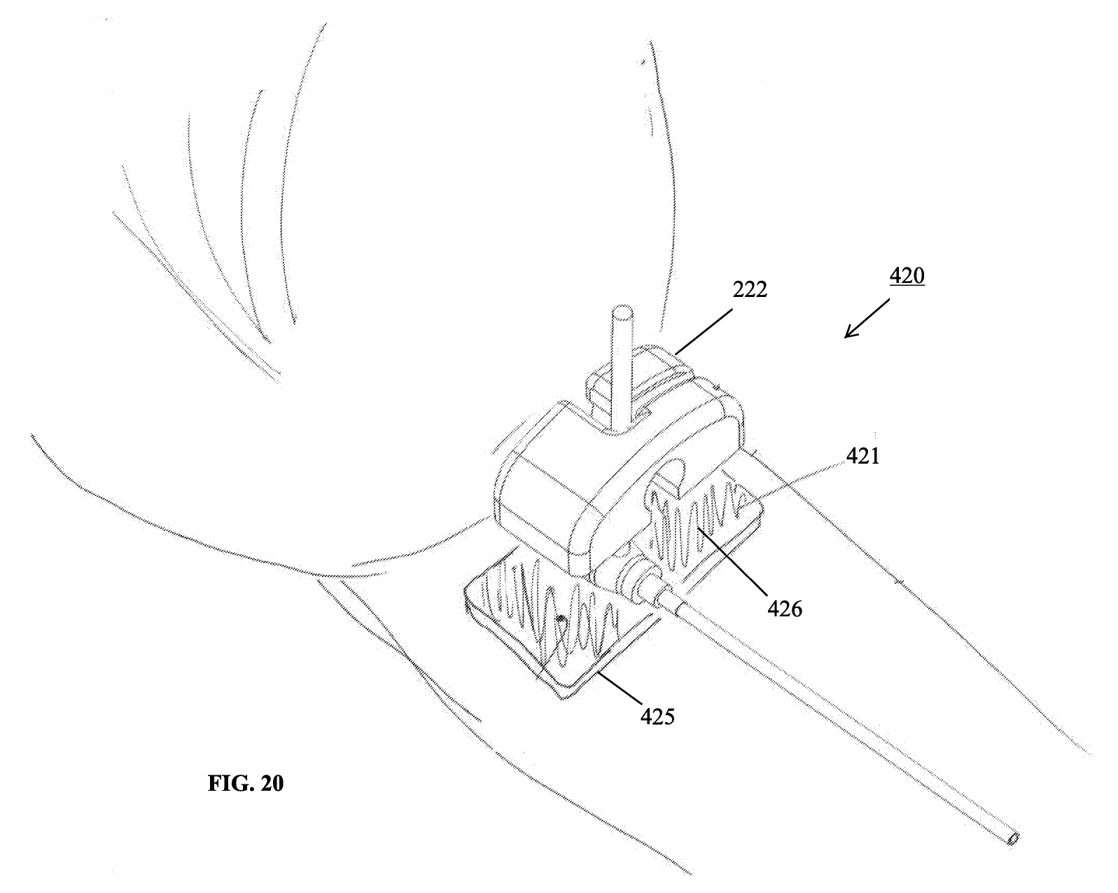

FIGS. 8 through 14 illustrate a securement device 220 including a retention pad 222 that may be manufactured in significant part of a polymeric material. The retention pad has an access slot 230, a side port receiver 224 and a contact surface 225. The contact surface has a retaining film thereon, such as an adhesive, a hydrogel, or a Velcro-type material that may be connected with an adhesive pad 421 shown in FIG. 20. Referring to FIG. 11, a sheath 10 is placed within a blood vessel and the hub 11 is located in the desired position on the anatomy. The sheath anchor 220 is placed over the hub by passing the hub through the access slot 230 and is adhered to the skin at the contact surface 225. After the anchor device is placed on the skin, the hub is rotated through an angle so that the side port 14 of the sheath engages the side port 224 of device 220 as seen in FIGS. 13 and 14. This engagement prevents axial movement of the sheath thereafter. Sheath interchange may be completed by rotating the hub 11 so that the side port 14 is free from the side port 224 of device 220, sliding the sheath out axially through the hub receiver 130 and thereafter replacing the sheath with another and re-engaging the side port in the side port receiver 120.

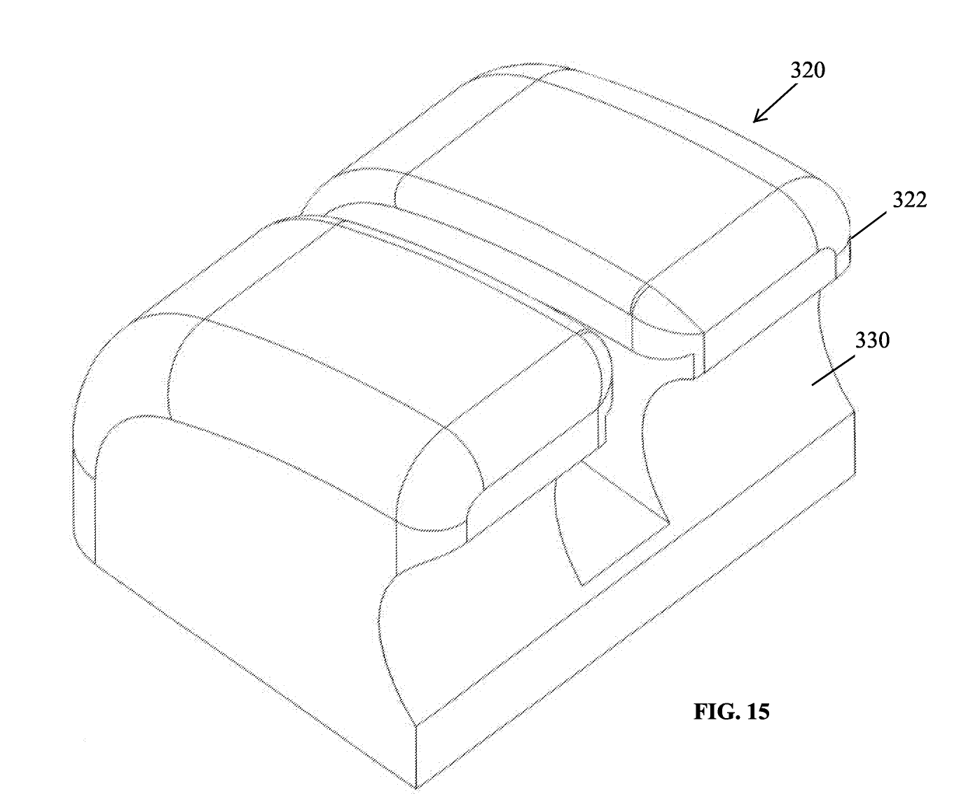

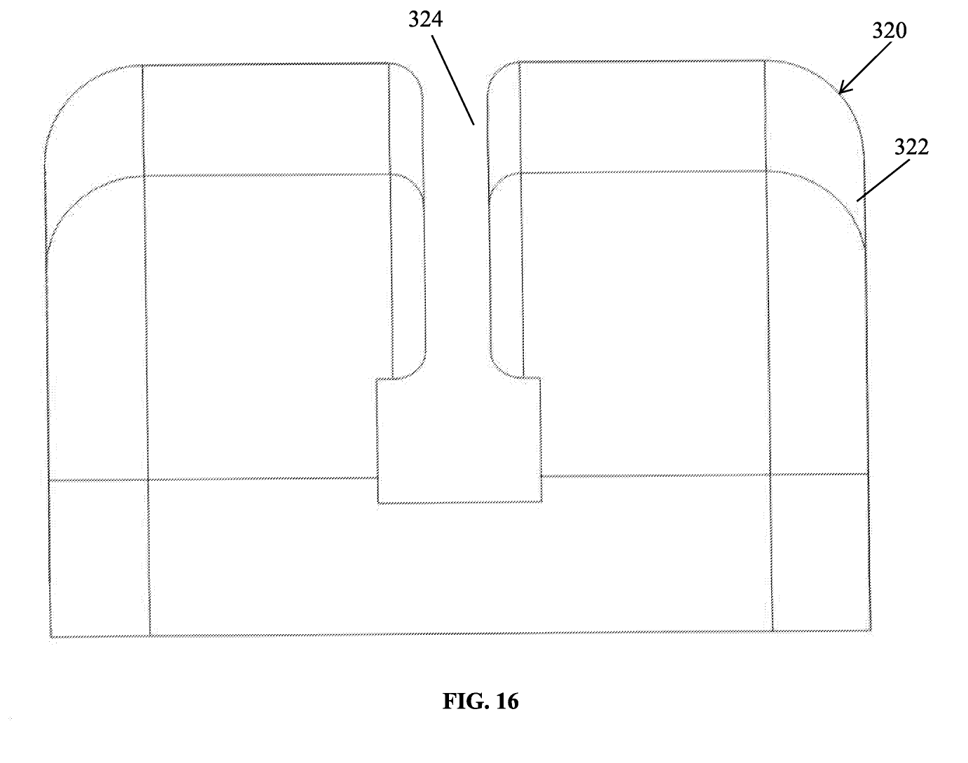

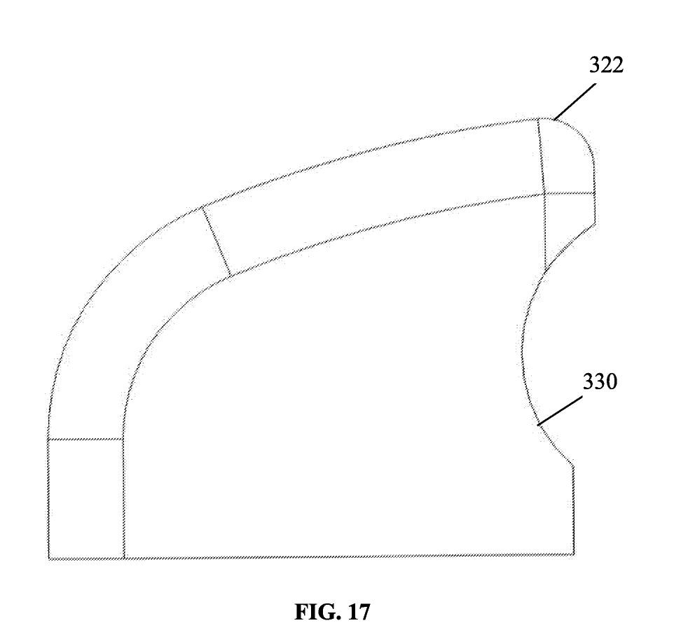

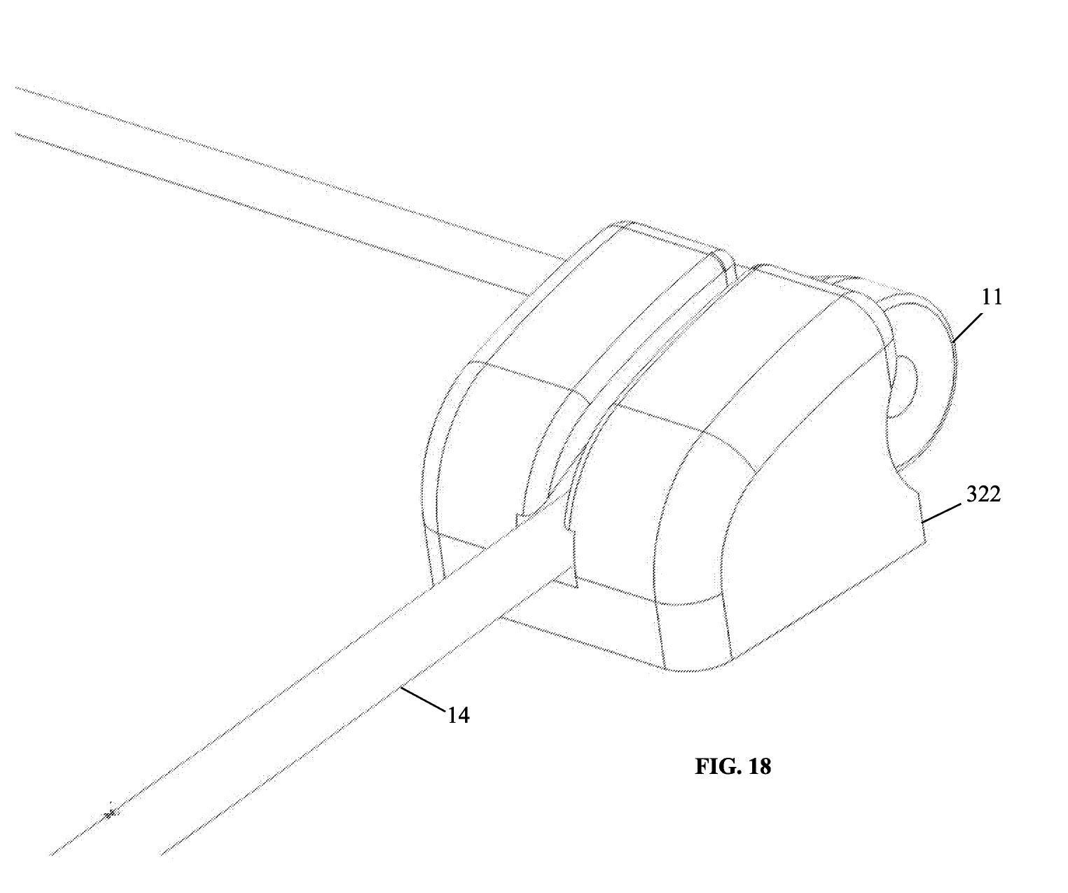

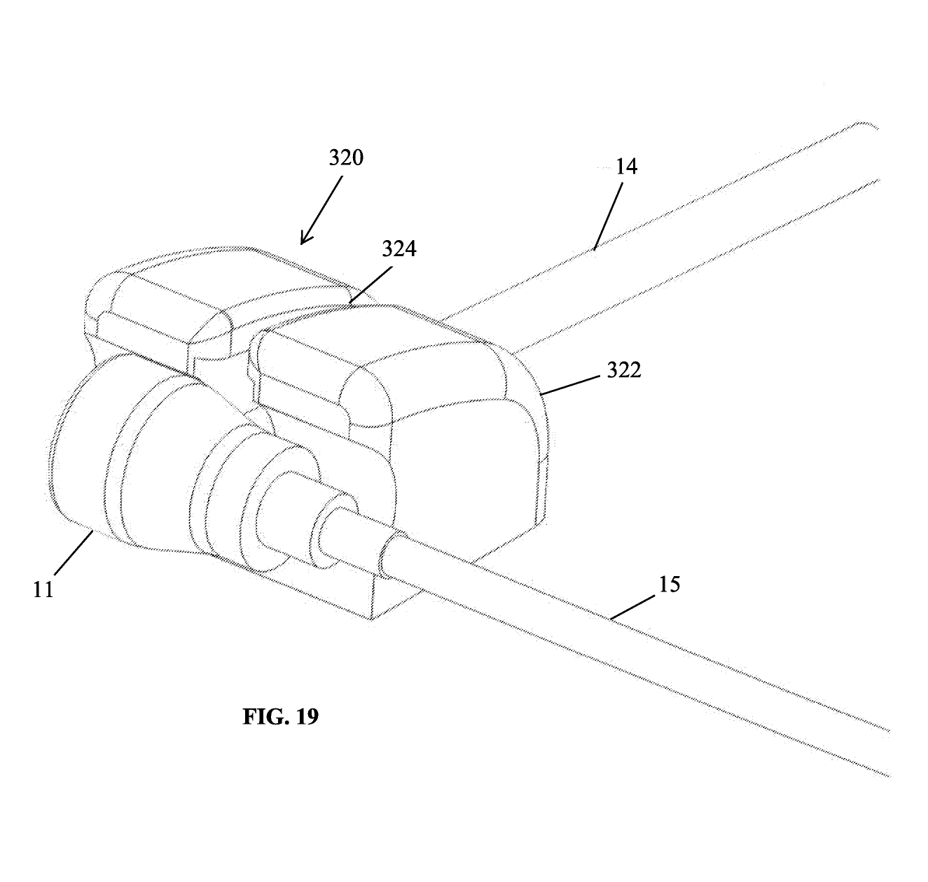

Referring to FIGS. 15 through 19 inclusive, an alternate embodiment of the securement device 320 is shown. A retention pad 322 has a receiver 324 for the side port of the sheath and allows unimpeded access to the hub of the sheath during the procedure.

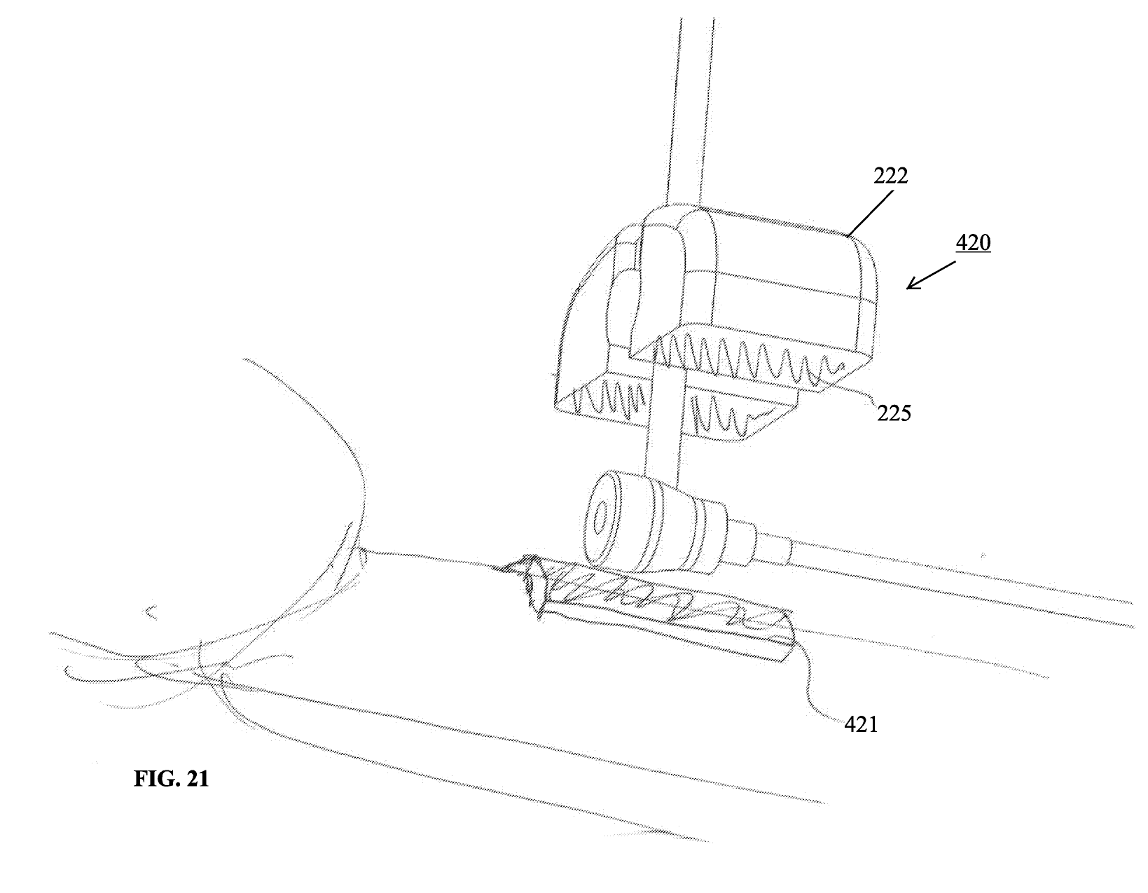

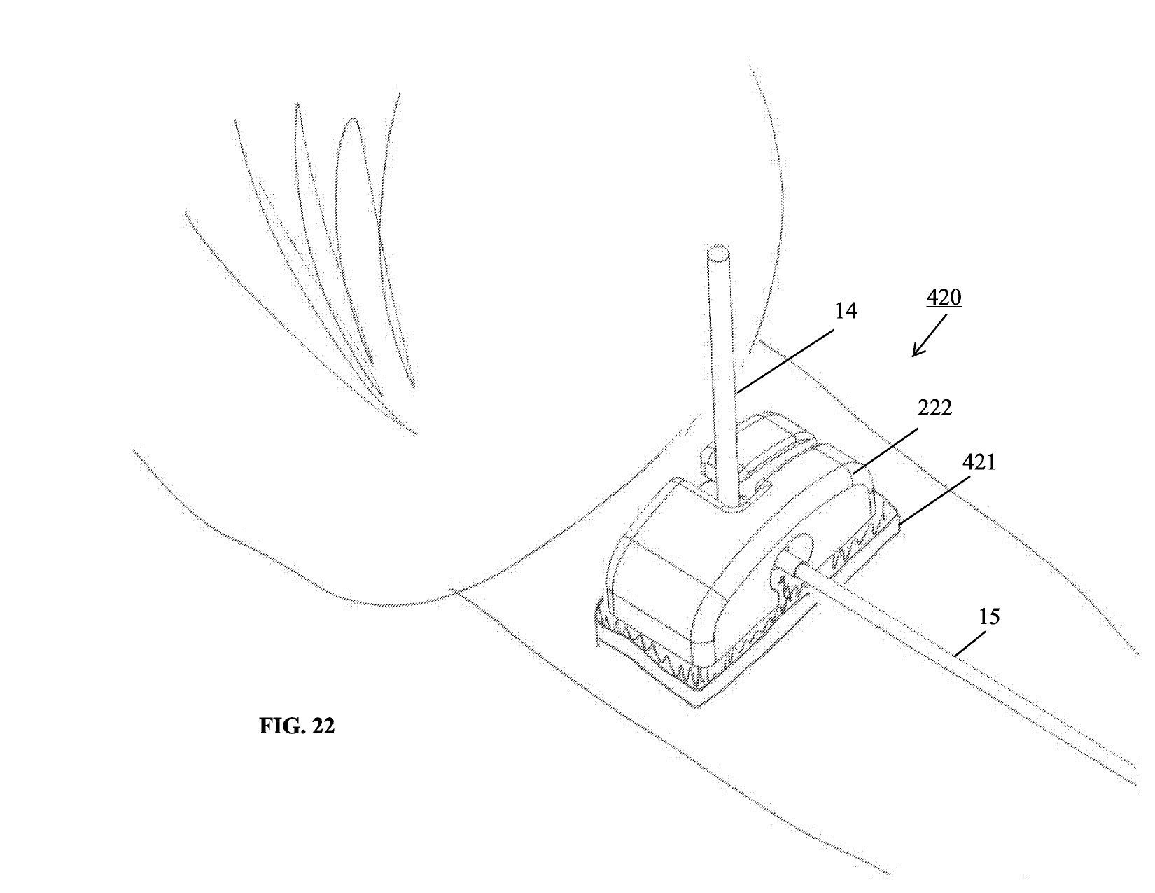

Referring now to FIGS. 20-22, securement device 420 is shown in use. Securement device 420 has a skin adhesive contact pad 421 which is adhered to the skin prior to making an incision or penetrating the blood vessel, thereby eliminating the issues associated with adhesion to wet or bloody skin. The skin contact pad 421 has a first surface 425 to contact and adhere to the skin by means of an adhesive layer and a second surface 426 having a surface which can adhere to the lower surface 225 of the retention pad 222. This may be a Velcro, hydrogel, adhesive, silicon, or other material. When the lower surface of the anchor 225 is brought into intimate contact with the contact surface 426 of the contact pad 421, the two components form an assembly. The components may be detachably engaged if the two mating surfaces are of detachable materials, such as Velcro, or may be permanently engaged if the materials are of permanent bonding types, such as adhesive tape or other materials. This construction has the advantage that the contact pad is placed and adhered to dry skin prior to placing the sheath assembly. Further, the construction allows the hub to be placed axially in the optimal position and anchored at this position thereafter. The locking mechanism of engaging the side port of the sheath by rotation into the anchor body is the same as previously described. Skin contact pad 421 could alternatively be used with retention pad 322.

While the foregoing description describes several embodiments of the present invention, it will be understood by those skilled in the art that variations and modifications to these embodiments may be made without departing from the spirit and scope of the invention, as defined in the claims below. The present invention encompasses all combinations of various embodiments or aspects of the invention described herein. It is understood that any and all embodiments of the present invention may be taken in conjunction with any other embodiment to describe additional embodiments of the present invention. Furthermore, any elements of an embodiment may be combined with any and all other elements of any of the embodiments to describe additional embodiments.

The embodiments of the invention in which an exclusive property or privilege is claimed are defined as follows:

1. An interventional cardiology access lumen site management device for use with an access lumen having a cylindrical hub and a tubular portion extending from said cylindrical hub, said site preparation device comprising:
an adhesive pad that is adapted to adhere directly or indirectly to a patient;
a retention pad supported with said adhesive pad, said retention pad having a surface configuration that is adapted to engage a portion of an access lumen; and
a top retention strip, said top retention strip adapted to retain an access lumen to the retention pad, wherein the site preparation device is adapted to adhere directly or indirectly prior to deployment of the access lumen and is adapted to secure the access lumen after deployment of the access lumen.

2. The site management device as claimed in claim 1 wherein said surface configuration is complementary to that of the hub.

3. The site management device as claimed in claim 2 for use with an access lumen wherein the hub of the access lumen is cylindrical and wherein the surface configuration has a partial circular shape.

4. The site management device as claimed in claim 1 including a side port receiver that is configured to the size and shape of a side port of the access lumen.

5. The site management device as claimed in claim 4 wherein said side port receiver includes side ribs that are configured to the size and shape of a side port of the access lumen.

6. The site management device as claimed in claim 5 wherein said side ribs extend laterally in opposite directions from the partial-circular shape to allow the side port of the access lumen to extend in either lateral direction.

7. The site management device as claimed in claim 1 wherein said retention pad and top retention strip are configured to provide access proximally to an insertion hole in the access lumen.

8. The site management device as claimed in claim 7 wherein said top retention strip comprises an adhesive strip.

9. The site management device as claimed in claim 7 wherein said top retention strip defines a through-channel that is adapted to pass an extension of said side port.

10. The site management device as claimed in claim 9 wherein said adhesive pad includes another through-channel that aligns with said through-channel in said top retention strip.

11. The site management device as claimed in claim 1 wherein said adhesive pad is configured to wrap at least partially around a patient's extremity to adhere directly to the patient.

12. The site management device as claimed in claim 1 wherein the adhesive pad is configured to wrap at least partially around a patient's wrist or ankle.

13. The site management device as claimed in claim 1 wherein said adhesive pad is configured to adhere to a drape at the intervention site to adhere indirectly to the patient.

14. A method of managing an access site for an interventional cardiology access lumen having a hub and a tubular portion extending from said cylindrical hub, said method comprising:
adhering an adhesive pad directly or indirectly to a patient at an intervention cardiology access site;
after said adhering, deploying an access lumen and positioning a portion of the access lumen against a retention pad that is supported by said adhesive pad, said retention pad having a surface configuration adapted to engage the portion of the access lumen; and
retaining the access lumen to the retention pad with a top retention strip.

15. The method of managing as claimed in claim 14 wherein said portion of the access lumen is the hub and wherein said surface configuration is complementary to that of the hub.

16. The method of managing as claimed in claim 15 wherein said hub is cylindrical and wherein said surface configuration has a partial-circular shape.

17. The method of managing as claimed in claim 14 including a side port receiver that is configured to the size and shape of a side port of the access lumen.

18. The method of managing as claimed in claim 17 wherein said side port receiver includes side ribs that are configured to the size and shape of a side port of the access lumen.

19. The method of managing as claimed in claim 18 wherein said side ribs extend laterally in opposite directions from the partial-circular shape in order to allow the side port of the access lumen to extend in either lateral direction.

20. The method of managing as claimed in claim 14 including providing proximal access to an insertion hole in the access lumen positioned against the retention pad and retained by the top retention strip.

21. The method of managing as claimed in claim 20 wherein said top retention strip comprises an adhesive strip.

22. The method of managing as claimed in claim 20 wherein said top retention strip defines a through-channel and including passing an extension of the side port through said channel.

23. The method of managing as claimed in claim 22 wherein said adhesive pad includes another through-channel that aligns with said through-channel in said top retention strip and wherein said passing includes passing said extension of the side port through said another channel.

24. The method of managing as claimed in claim 14 including adhering said adhesive pad directly to the patient by wrapping the adhesive pad at least partially around a patient's extremity.

25. The method of managing as claimed in claim 24 wherein the patient's extremity is the wrist or ankle.

26. The method of managing as claimed in claim 24 including adhering said adhesive pad indirectly to the patient by adhering the adhesive pad to a drape at the intervention site.

27. The method of managing as claimed in claim 14 including providing access to an insertion opening at the hub of the retained access lumen.

28. An interventional cardiology access lumen site management device that is adapted to adhere directly or indirectly prior to deployment of the access lumen for use with an access lumen having a cylindrical hub and a tubular portion extending from said cylindrical hub, comprises:
an adhesive pad that is adapted to adhere directly or indirectly to a patient;
a retention pad supported with the adhesive pad, the retention pad having a surface configuration that is adapted to engage a portion of an access lumen and adapted to retain the access lumen; and
said retention pad having a side port receiver that is configured to the size and shape of a side port of the access lumen.
